(19) United States
(12) Patent Application Publication
(10) Pub. No.: US 2005/0019291 A1
(43) Pub. Date: Jan. 27, 2005

(54) Emulsion composition of polyvinyl alcohol which forms a peelable film on skin

(76) Inventors: Yelena Zolotarsky, Springfield, NJ (US); Cary Lamont Sims, Iselin, NJ (US); Ellen S. Botschka, Riverdale, NJ (US); Barbara Krautsieder, Elmwood Park, NJ (US)

Correspondence Address: William J. Davis, Esq.;INTERNATIONAL SPECIALTY PRODUCTS, Legal Dept., Bldg. 10, 1361 Alps Road, Wayne, NJ, 07470-3529, US

(21) Appl. No.: 10/626,452

(22) Filed: Jul. 24, 2003

(51) Int. Cl. A61K 7/06; A61K 7/11

(52) U.S. Cl. 424/70.11

(57) ABSTRACT

An emulsion composition which forms a readily peelable polyvinyl alcohol film upon drying on skin, includes (a) polyvinyl alcohol, (b) a thickener, and (c) water, optionally with one or more oil or water soluble or dispersible ingredients.

### BACKGROUND OF THE INVENTION

1. Field of the Invention

This invention relates to polymeric films, and, more particularly to an emulsion composition of polyvinyl alcohol (PVA) which forms a thin, soft, flexible and readily peelable PVA film on skin.

2. Description of the Prior Art

Sheet-like material for the treatment of skin, e.g. face masks, ordinarily are produced from gels of polyvinyl alcohol; see EP 309309 (1989). However, such gel compositions do not allow for ready modification of the feel of the masks by inclusion therein of such cosmetic materials as emollients and oils, and other oil soluble or dispersible ingredients.

Accordingly, it is an object of this invention to provide a polyvinyl alcohol-based emulsion composition which forms a thin, soft, flexible and readily peelable PVA-film upon drying on skin, and which film has advantageous physical and feel characteristics for the user.

These and other objects and features of the invention will be made apparent from the following description of the invention.

### SUMMARY OF THE INVENTION

What is described herein is an emulsion composition of polyvinyl alcohol (PVA) which forms a thin, soft, flexible and readily peelable PVA film upon drying on skin. The emulsion composition of the invention includes (a) polyvinyl alcohol, (b) a thickener, and (c) water, optionally with one or more oil or water soluble or dispersible ingredients.

Suitably, by weight, the emulsion composition includes (a) 3-20%, preferably 5-15%; (b) 0.5-10%, preferably 0.8-3%; and (c) the rest.

Typically the optional ingredients include such active ingredients as cosmetic and/or drug compounds, and/or such additional materials as pigments, beads, silicones and/or oils and esters, including natural or synthetic compounds.

Preferably, the emulsion composition of the invention has a pH of about 3-8; and its Brookfield viscosity is 10,000 to 300,000 cps (RTC @ 10 rpm), most preferably 20,000 to 80,000 cps.

A typical thickener, (b) is a sodium polyacrylate, or a polyacrylamide, or mixtures of each of both, dispersed in an oil and an emulsifier.

In addition to water, other solvents may be included in the emulsion composition, e.g. mono-alcohols, such as ethanol; poly-alcohols, e.g. glycols such as ethylene glycol, and/or fluoro- and hydrocarbons.

### DETAILED DESCRIPTION OF THE INVENTION

The examples which follow herein illustrate PVA-based emulsion compositions of the invention which form PVA-films that are readily peelable upon drying on skin. The films obtained herein have an advantageous feel for the user and are thin, soft and flexible. These film properties are particularly suitable for use as a face mask, or, in a patch on skin, to deliver an active ingredient, e.g. a cosmetic and/or a drug. Polyvinyl alcohol is present in the emulsion composition of the invention in an amount of about 3-20 wt. %, preferably 5-15 wt. %.

A typical thickener is RapiThix™ A-60 (International Specialty Products) which is a cold mix thickener composed of sodium polyacrylate (60 parts) as viscosity enhancer, an oil, (30-38 parts) and an emulsifier, (4-8 parts). Suitably (b) is present in the emulsion composition of the invention in an amount of about 0.5-10 wt. %, preferably 0.8-5 wt. %.

Water is added to form the desired emulsion. Other solvents may be included either to enable faster drying, e.g. monoalcohol or poly alcohols, or to act as a plasticizer.

Optional ingredients include one or more water soluble or dispersible materials, e.g. active ingredients such as cosmetic or drug compounds; as well as pigments, beads, silicones and natural or synthetic oils or esters.

### EXAMPLE 1

### Ester Based PVA-Emulsion Composition

| Phase | Ingredient | % W/W |
|---|---|---|
| A | DI water | 71.72 |
| | Na₂EDTA | 0.05 |
| | PVA (Celvol ® 525) Celanese | 8.00 |
| B | RapiThix ™ A-60 (ISP) | 1.73 |
| | Glycerin | 3.00 |
| C | Ceraphyl ® 368 (ISP) | 3.00 |
| | Ceraphyl ® 494 (ISP) | 2.00 |
| D | EtOH | 10.00 |
| | LGP | 0.50 |
| | Total: | 100.00 |

Procedure
1. Disperse EDTA in water. Slowly add PVA and heat to 85° C. Maintain at 85° for 30 minutes; then turn-off heat.
2. Add RapiThix A-60™ and allow to completely disperse. Add glycerin.
3. Combine C and add to A and B with agitation. Add D individually @ 35° C. QS with water.

The PVA-emulsion composition has a Brookfield viscosity of 92,000 cps (RTC @ 10 rpm) and a pH of 6. When formed into a face mask and dried, a thin, soft, flexible PVA film was present on the skin of the user. The mask was readily peelable and could be completely removed after use.

### EXAMPLE 2

### Silicone-Based PVA-Emulsion Composition of Invention

| Phase | Ingredient | % W/W |
|---|---|---|
| A | DI water | 71.22 |
| | Na₂EDTA | 0.05 |
| | PVA | 8.00 |
| B | RapiThix ™ A-60 | 1.73 |
| | Glycerin | 3.00 |
| C | Si Tec PTM 20 | 1.50 |
| | Si Tec LDM 3107 | 0.75 |
| | Si Tec DM 100 | 3.25 |
| D | EtOH | 10.00 |
| | LGP | 0.50 |
| | Total: | 100.00 |

Procedure
1. Disperse EDTA in water. Slowly add PVA and heat to 85° C. Maintain at 850 for 30 minutes; then turn-off heat.
2. Add RapiThix™ and allow to completely disperse. Add glycerin.
3. Combine C and add to A+B with agitation. Add D individually @ 35° C. QS with water.

The composition has a pH of 6.01 and a Brookfield viscosity of 80,800 cps, (RTC @ 10 rpm). Readily peelable PVA-films were obtained.

### EXAMPLE 3

### Sunscreen-Based PVA-Emulsion Composition of Invention

| Phase | Ingredient | % W/W |
|---|---|---|
| A | DI water | 33.17 |
| | Na₂EDTA | 0.05 |
| | LGP | 0.25 |
| | Glycerin | 3.00 |
| | RapiThix ™ A-60 | 1.73 |
| | PVA (20% soln) | 45.00 |
| B | Escalol ® 557 (ISP) | 3.80 |
| | Escalol ® 567 (ISP) | 1.50 |
| | Escalol ® 587 (ISP) | 1.50 |
| C | EtOH | 10.00 |
| | Total: | 100.00 |

Procedure
1. Add phase A ingredients in order listed with good mixing between additions.
2. Combine phase B and heat to 45-50° C. When uniform, add to A with good mixing.
3. Add C to A+B. QS with water.

The emulsion composition has a pH of 5.9 and a Brookfield viscosity of 202,000 cps.

### EXAMPLE 4

### PVA-Pearl Mask Emulsion Composition of Invention

| Phase | Ingredient | % W/W |
|---|---|---|
| A | Deionized Water | 70.22 |
| | Disodium EDTA | 0.05 |
| | Glycerin | 3.00 |
| | PVA | 8.00 |
| B | RapiThix ™ A-60 | 1.73 |
| C | Ceraphyl ® 368 | 3.00 |
| | Ceraphyl ® 494 | 2.00 |
| D | Liquid Germall Plus ® (ISP) | 0.50 |
| | SD 40B Alcohol | 5.00 |
| E | Cloisonne ® Super Rouge | 6.50 |
| | (Engelhard) | |
| | Total: | 100.00 |

Procedure:
1. Add EDTA and glycerin to water at room temperature; mix until uniform.
2. Add PVA with vigorous agitation. Begin heating to 85-90° C.
3. Hold temperature for 30 minutes. Turn off heat.
4. Begin slow cooling to 50° C.; add Phase B to batch, allow to fully disperse.
5. Premix Phase C and add to batch at 50° C. and mix until uniform.
6. Cool batch to 30° C. while stirring slowly. Add Phase D ingredients one at a time; mix until uniform.
7. At room temperature, add Phase E slowly, avoid aerating batch, mix until uniform.

The emulsion composition has a pH of 5.8 and a Brookfield viscosity of 174,000 cps (RTC, 5 rpm).

While the invention has been described with particular reference to certain embodiments thereof, it will be understood that changes and modifications may be made which are within the skill of the art. Accordingly, it is intended to be bound only by the following claims, in which:

1. An emulsion composition of polyvinyl alcohol (PVA) which forms a thin, soft, flexible and readily peelable PVA film upon drying on skin, comprising (a) polyvinyl alcohol, (b) a thickener, and (c) water, optionally with one or more oil or water soluble or dispersible ingredients.

2. An emulsion composition according to claim 1 wherein, by weight, (a) is 3-20%, (b) is 0.5-10%, and (c) the rest.

3. An emulsion composition of claim 2 wherein (a) is 5-15%, and (b) is 0.8-5%.

4. An emulsion composition of claim 1 wherein said optional ingredients include active, cosmetic and drug compounds, and/or pigments, beads, silicones, oils, natural or synthetic, and esters.

5. An emulsion composition of claim 1 which has a pH of about 3-8.

6. An emulsion composition of claim 1 which has a Brookfield viscosity of 10,000 to 300,000 cps (RTC @ 10 rpm).

7. An emulsion composition according to claim 6 wherein said viscosity is 20,000 to 80,000 cps.

8. An emulsion composition according to claim 1 wherein (b) is a sodium polyacrylate, or a polyacrylamide, or mixtures of each or both, dispersed in an oil and an emulsifier.
